(19) United States
(12) Patent Application Publication
(10) Pub. No.: US 2003/0186384 A1
(43) Pub. Date: Oct. 2, 2003

(54) Apoptotic agents

(76) Inventors: Stefan Barth, Roetgen (DE); Andreas Engert, Koln (DE); Michael Stocker, Koln (DE)

Correspondence Address: JACOBSON HOLMAN PLLC, 400 SEVENTH STREET N.W., SUITE 600, WASHINGTON, DC, 20004, US

(21) Appl. No.: 10/257,931

(22) PCT Filed: Apr. 20, 2001
(86) PCT No.: PCT/EP01/04514

(30) Foreign Application Priority Data
Apr. 22, 2000 (DE) 100 20 095.8

(51) Int. Cl. C07H 21/04; C12P 21/02; A61K 38/48; A61K 39/395; C12N 9/64; A61K 48/00; C12N 1/21; C12N 15/74

(52) U.S. Cl. 435/69.5; 435/69.7; 435/226; 435/320.1; 435/252.3; 424/94.63; 514/44; 536/23.2; 424/178.1; 424/85.1

(57) ABSTRACT

A complex at least formed from at least one component A and at least one component B, wherein component A has a binding activity for cellular surface structures, and component B carries a protease or derivatives thereof as an effector function.

The present invention relates to a heterologous, chemically coupled or recombinantly prepared complex which comprises at least one proteolytic domain and one cell-specific binding domain, especially of human origin, and nucleic acids and vectors coding for such a complex. It further relates to methods for influencing cell growth and the physiology of cells with the complex according to the invention or with vectors containing the nucleic acid coding therefor. The invention further relates to vectors and hosts for producing the complex according to the invention. It further relates to the preparation and distribution of medicaments based on the complex according to the invention or vectors coding therefor, for the treatment of diseases based on a pathological proliferation and/or increased activity of structurally defined cell populations. This applies, in particular, to tumor diseases, allergies, autoimmune diseases, chronic inflammation reactions, or tissue rejection reactions.

In the medicamentous treatment of tumors, autoimmune diseases, allergies and tissue rejection reactions, it is a disadvantage that the currently available medicaments, such as chemotherapeutic agents, corticosteroids and immunosuppressive agents, have a potential of side effects which is sometimes considerable, due to their relative non-specificity. It has been attempted to moderate this by various therapeutical concepts. Especially the use of immunotherapeutic agents is an approach which resulted in an increase of the specificity of medicaments, especially in tumor treatment.

If the immunotherapeutic agent is an immunotoxin, then a monoclonal antibody (moAb) or an antibody fragment which has a kinetic affinity for surface markers of tumor cells is coupled with a cytotoxic reagent. If the immunotherapeutic agent is an anti-immunoconjugate for the treatment of autoimmune diseases, tissue rejection reactions or allergies, a structure relevant to pathogenesis or a fragment thereof is coupled to a toxin component. It has been found that immunotoxins can be characterized by a high immunogenicity in clinical use. This causes the formation of neutralizing antibodies in the patient which inactivate the immunotoxin. Generally, a repeated and/or continuous administration of the therapeutic agents is unavoidable for long-term curative effects. This is particularly clear in the suppression of tissue rejection reactions after transplantations, or in the treatment of autoimmune diseases, due to the partly demonstrated genetically caused predisposition to a pathogenic autoimmune reaction.

Recombinant Fusion Proteins Based on Autologous Apoptosis-Inducing Proteases (Apoptotic Agents)

To achieve a direct therapeutic effect on the target cells, antibodies were linked with radioactive elements or toxins to form so-called radioimmunoconjugates or immunotoxins (ITs). When radioactively labeled anti-B-cell moAb were used with B-cell lymphomas, tumor regressions and even complete remissions could be observed (Jurcic, J. G. and Scheinberg, D. A. 1995; Kaminski, M. S. et al. 1996; Press, O. W. et al. 1993). In contrast, the results with moAb against solid tumors were rather disillusioning (LoBuglio, A. F. and Saleh, M. N. 1992; Saleh, M. N. et al. 1992). An explanation thereof seems to be the too low tumor penetration due to their size, especially for poorly vascularized tumors. Therefore, in the further development, antibody fragments or target-cell-specific ligands were coupled to the corresponding effectors. The reasons for the miniaturization were a better tissue and tumor penetration by improved diffusion properties, and a hoped-for lower immunogenicity due to the reduction of the antigenic determinants (Pirker, R. 1988; Yokota, T. et al. 1992). Improved cloning techniques enabled the completely recombinant preparation of ITs. Thus, the pieces of genetic information of the variable domains of a moAb are linked to one another through a synthetic (Gly₄Ser)₃ linker to give a single-stranded fragment (scFv). Through another fusion on the DNA level, the catalytic domain, such as of a toxin, is fusioned to the scFv (Chaudhary, V. et al. 1990; Chaudhary, V. et al. 1989). In addition to the use of scFv, ligands for tumor-cell-specific receptors may also be coupled to the toxins (Klimka, A. et al. 1996). In addition to such active binders, passive binding structures may also be employed for cell-specific targeting. The essential difference is based on the fact that immunoglobulins, such as antibodies and T-cell receptors, “recognize” auto-antigens and allergens. Thus, if the immunotherapeutic agent is an anti-immunoconjugate for the treatment of autoimmune diseases, tissue rejection reactions or allergies, a structure relevant to pathogenesis or a fragment thereof is coupled to a toxin component (Brenner, T. et al. 1999).

The peptidic cell poisons which have been mostly used to date and are thus best characterized are the bacterial toxins diphtheria toxin (DT) (Beaumelle, B. et al. 1992; Chaudhary, V. et al. 1990; Kuzel, T. M. et al. 1993; LeMaistre, C. et al. 1998), Pseudomonas exotoxin A (PE) (Fitz Gerald, D. J. et al. 1988; Pai, L. H. and Pastan, I. 1998), and the plant-derived ricin-A (Engert, A. et al. 1997; Matthey, B. et al. 2000; O'Hare, M. et al. 1990; Schnell, R. et al. 2000; Thorpe, P. E. et al. 1988; Youle, R. ]. and Neville, D. M. 3. 1980). The mechanism of cytotoxic activity is the same in all of these toxins despite of their different evolutionary backgrounds. The catalytic domain inhibits protein biosynthesis by a modification of the elongation factor EF-2, which is important to translation, or of the ribosomes directly, so that EF-2 can no longer bind (Endo, Y. et al. 1987; Iglewski, B. H. and Kabat, D. 1975).

In most of the constructs employed to date, the systemic application of immunotoxins results in more or less strong side effects. In addition to the “vascular leak” syndrome (Baluna, R. and Vitetta, E. S. 1997; Schnell, R. et al. 1998; Vitetta, E. S. 2000), thrombocytopenia, hemolysis, renal insufficiency and sickness occur, depending on the construct employed and the applied dosage. Dose-dependent and reversible liver damage could also be observed (Battelli, M. G. et al. 1996; Grossbard, M. L. et al. 1993; Harkonen, S. et al. 1987). In addition to the documented side effects, the immunogenicity of the constructs employed to be observed in the use of the immunoconjugates or immunotoxins is the key problem of immunotherapy (Khazaeli, M. B. et al. 1994). This applies, in particular, to the humoral defense against the catalytic domains employed, such as ricin (HARA) (Grossbard, M. L. et al. 1998), PE (Kreitman, R. J. et al. 2000), or DT (LeMaistre, C. F. et al. 1992). Theoretically, all non-human structures can provoke an immune response. Thus, the repeated administration of immunotoxins and immunoconjugates is subject to limitations. A logical consequence of these problems is the development of human immunotoxins (Rybak, S. et al. 1992).

To date, human toxins for use in immunotoxins have been selected exclusively from so-called ribonucleases. After the cytotoxic potential of human RNase A could be shown by microinjection into cells (Rybak, S. et al. 1991), it was chemically coupled to an anti-CD5 moAb and successfully tested in an in-vivo model (Newton, D. L. et al. 1992). Since human RNases are present in extracellular fluids, plasma and tissues, they are considered to be less immunogenic when used in immunotoxins. Angiogenin (ANG), a 14 kDa protein having a 64% sequence homology with RNase A, was first isolated from a tumor-cell-conditioned medium, where it was discovered due to its capability of inducing angiogenesis (Fett, J. W. et al. 1985). It could be shown that the t-RNA-specific RNase activity of angiogenin has a cytotoxic potential (Saxena, S. K. et al. 1992; Shapiro, R. et al. 1986). Correspondingly chemically conjugated immunotoxins subsequently exhibited a cell-specific toxic activity (Newton, D. et al. 1996; Yoon, J. M. et al. 1999). To evaluate the effectiveness of ANG-based scFv immunotoxins, different conformations of ANG with epidermal growth factor (EGF) were constructed and successfully tested in vitro (Yoon, J. M. et al. 1999). Another member of the RNase superfamily is eosinophilic neurotoxin (EDN). For EDN, which has a size of 18.4 kDa, only the direct neurotoxicity could be described to date. On the basis of the documented potency, different EDN-based immunotoxins were constructed and also successfully tested in vitro (Newton, D. et al. 1994; Zewe, M. et al. 1997).

More recent studies have shown that ANG can be blocked by an endogenous cytoplasmic ribonuclease inhibitor (RI). This limits the effectiveness of ANG-based ITs in RI(+) target cells (Leland, P. A. et al. 1998).

The invention is based on the following objects:

Reduction of the immunogenicity of the immunotherapeutic agents, decrease of activity reduction by non-specific inactivation, and improvement of the activity which is reduced by endogenous specific inhibitors.

These objects are achieved by a complex which is formed from at least one component A and at least one component B, wherein component A has a binding activity for cellular surface structures, and component B carries a protease or derivatives thereof as an effector function.

The complex according to the invention can be regarded as a heterologous complex which comprises at least two domains, i.e., one effector domain and one binding domain.

The effector domain consists of a protease endogenous to the organism to be treated, preferably granzyme B in humans, a protease inducing natural apoptosis or a derivative thereof. The binding domain consists of a structure which enables binding to and internalization into structurally defined target cells.

It is advantageous that the catalytic domain is an endogenous protein or a derivative thereof and as a result thereof, that the immunogenicity to be expected is drastically reduced. Especially the reactive cells of the immune system, which are to be eliminated in connection with autoimmune diseases, allergies and tissue rejection reactions, are normal cells in a physiological sense. With these cells, a normal sensitivity towards natural apoptosis-inducing elements can be expected.

Preferably, the complex according to the invention has one or more supplementary components S in addition to components A and B. From his former experience, the skilled person knows that additional features and properties can have a critical importance to the efficient preparation and/or effectiveness of the complexes according to the invention. Due to the distinctness of the diseases to be treated with the complexes according to the invention, an adaptation of the complexes to the respective particular circumstances may be necessary.

Preferably, component A of the complex according to the invention is selected from the group of-actively binding structures consisting of antibodies, their derivatives and/or fragments, synthetic peptides or chemical molecules, ligands, lectins, receptor binding molecules, cytokines, lymphokines, chemokines, adhesion molecules, which bind to cluster of differentiation (CD) antigens, cytokine, hormone, growth factor receptors, ion pumps, channel-forming proteins, and their derivatives, mutants or combinations thereof.

In another embodiment of the complex according to the invention, it is characterized in that component A is selected from the group of passively bound structures consisting of allergens, peptidic allergens, recombinant allergens, allergen-idiotypical antibodies, autoimmune-provoking structures, tissue-rejection-inducing structures and their derivatives, mutants or combinations thereof.

Component B of the complex according to the invention has, in particular, proteolytic properties or at least one protease, its derivatives, mutants or combinations thereof.

None of the effector domains described to date in immunotoxins use proteolytic properties and directly initiate the natural mechanisms for inducing apoptosis in the target cells. The effects of the immunotoxins described to date are always based on a disorder or inhibition of translation in the target cells. The resulting adverse affection of the vitality of the cells can indirectly lead to the initiation of apoptosis (Bolognesi, A. et al. 1996; Keppler-Hafkemeyer, A. et al. 1998; Keppler-Hafkemeyer, A. et al. 2000). Preferably, component B of the complex according to the invention directly activates components of cell-inherent apoptosis and thus induces apoptosis in the cells defined through the binding of component A.

In another embodiment of the complex according to the invention, component B is a member of the cathepsin protease family, of the calpains, granzymes, or a derivative of the above mentioned proteins, or a combination thereof.

Particularly preferred as component B of the complex according to the invention is granzyme B (GB) or a derivative thereof. The serine-dependent and aspartate-specific protease granzyme B is of particular interest. Granzyme B is a component of cellular immune defense which, upon activation of cytotoxic T cells (CTL) or natural killer cells (NK), is secreted from the cytotoxic granules of these cells (Kam, C. M. et al. 2000; Shresta, S. et al. 1998). Upon the perforin-dependent translocation of granzyme B into the cytoplasm of attacked cells, a proteolytic cascade is initiated which ends in the apoptosis of the target cell (Greenberg, A. H. 1996). The exact function of the perforin secreted along with granzyme B is still being discussed currently, but it is not capable of inducing apoptosis alone. In the cell membrane, perforin aggregates into 12-18 mers and thereby forms pores of 15-18 nm. Initially, it was considered that granzyme B gets into the cytoplasm of the target cells through these pores. However, the 32 kDa protein granzyme B is too large for such a passage. It is more probable to assume that, after granzyme B has bound to perforin and this complex is successively internalized, perforin supports the endosomal release of granzyme B (Jans, D. A. et al. 1996). In recent years, various proteins could be identified which are activated by GB-mediated cleavage are directly related to apoptosis. Thus, the GB-caused proteolytic activation of various procaspases, especially 3 and 8, could be documented in vitro (Fernandes-Alnemri, T. et al. 1996; Srinivasula, S. M. et al. 1996); these are counted with the central proteases in apoptosis (Nicholson, D. W. and Thornberry, N. A. 1997). Further cytotoxic activities are displayed by granzyme B in the nucleus. After having intruded the cytoplasms of the target cell, granzyme B is relatively quickly translocated into the nucleus in a caspase-dependent way (Pinkoski, M. J. et al. 2000). There, granzyme B is capable, for example, of cutting nuclear matrix antigen and poly (ADP-ribose) polymerase (Andrade, F. et al. 1998). A quick apoptosis could be observed in cells after granzyme B accumulated in the nucleus (Trapani, J. A. et al. 1998; Trapani, J. A. et al. 1998). More recent data prove the initiation of apoptosis through the direct proteolytic cleavage of Bid, a member of the Bcl-2 family having only one BH3 domain. After cleavage, the truncated form tBid becomes embedded in the mitochondrial membrane and depolarizes it. This induces the release of cytochrome c and an apoptosis-inducing factor from the mitochondria into the cytoplasm, which critically accelerated cell death (Sutton, V. R. et al. 2000). Further caspase-independent toxic properties of granzyme B could be described, the underlying mechanism- still being uncleared (Beresford, P. J. et al. 1999; Sarin, A. et al. 1997).

Further embodiments of the complexes according to the invention can contain one or more different components S. Due to his knowledge, the skilled person is capable of evaluating the advantages and necessity of additional components and/or features in connection with the complexes according to the invention. The components S may serve the following purposes, for example:

the inducible regulation of synthetic performance (e.g., inducible promoters;

control of protein biosynthesis (e.g., leader sequence);

purification of the complex or its components (e.g., His tag, affinity tags);

translocation of the apoptotic agents into the target cells (e.g., translocation domain, amphiphatic sequences);

intracellular activation of component B (synthetic pro-granzyme B, amphiphatic sequences).

The invention also relates to nucleic acid molecules or vectors which code for the complex according to the invention or for individual components for preparing the complex. The inventors successfully documented the expression of the apoptotic agents in eukaryotic cells of human origin. This suggests the suitability of nucleic acids coding for a complex according to the invention also for gene-therapeutic approaches. Due to his knowledge, the skilled person is capable of recognizing the various aspects and possibilities of gene-therapeutic interventions in connection with the various diseases to be treated. In addition to the local application of relatively non-specific vectors (e.g., cationic lipids, non-viral, adenoviral and retroviral vectors), a systemic application with modified target-cell-specific vectors will also become possible in the near future. Until such systems are available, the well-aimed ex-vivo transfection of defined cell populations and their return into the organism to be treated offers an interesting alternative (Chen, S. et al. 1997).

Cellular compartments or organisms which synthesize complete complexes according to the invention or individual components thereof after transformation or transfection with the nucleic acid molecules or vectors according to the invention are also claimed according to the invention.

The cellular compartments according to the invention are of either prokaryotic origin, especially from E. coli, B. subtilis, S. carnosus, S. coelicolor, Marinococcus sp., or eukaryotic origin, especially from Saccharomyces sp., Aspergillus sp., Spodoptera sp., P. pastoris, primary or cultivated mammal cells, eukaryotic cell lines (e.g., CHO, Cos or 293) or plant systems (e.g. N. tabacum).

The invention also relates to medicaments containing a complex according to the invention. Typically, the complexes according to the invention are administered in physiologically acceptable dosage forms. These include, for example, Tris, NaCl, phosphate buffers and all approved buffer systems, especially including buffer systems which are characterized by the addition of approved protein stabilizers. The administration is effected, in particular, by parenteral, intravenous, subcutaneous, intramuscular, intratumoral, transnasal administrations, and by transmucosal application.

The dosage of the complexes according to the invention to be administered must be established for each application in each disease to be newly treated by clinical phase I studies (dose-escalation studies).

Nucleic acids or vectors which code for a complex according to the invention are advantageously administered in physiologically acceptable dosage forms. These include, for example, Tris, NaCl, phosphate buffers and all approved buffer systems, especially including buffer systems which are characterized by the addition of approved stabilizers for the nucleic acids and/or vectors to be used. The administration is effected, in particular, by parenteral, intravenous, subcutaneous, intramuscular, intratumoral, transnasal administrations, and by transmucosal application.

The complex according to the invention, nucleic acid molecules coding therefor and/or cellular compartments can be used for the preparation of a medicament for treating malignant diseases, allergies, autoimmune reactions, chronic inflammation reactions or tissue rejection reactions.

For the example of the anti-CD30 apoptotic agent Ki-4 (scFv)-granzyme B (see below) (KGbMH), the cytotoxic effectiveness of a complex based on the present invention could be proven for the example of the Hodgkin cell line L540Cy. The secretion of this functional complex from eukaryotic cells additionally demonstrates the potential suitability of the proteins according to the invention for a gene-therapeutic application.

Preparation of the Recombinant CD30-Specific Apoptotic Agent Ki-4(scFv)-Granzyme B (KGbMH)

Methods

Bacteria, Oligonucleotides and Plasmids

E. coli XL1-blue was used for the propagation of the plasmids. Synthetic oligonucleotides were acquired from the company MWG (Martinsried, Germany). The preparation of the plasmids was performed according to the alkaline lysis method, and the plasmids were purified by means of the plasmid purification kits from Qiagen (Hilden, Germany).

Cell Lines

All cell lines employed (Table 1) were cultured in a complex medium (RPMI-1640, 10% FCS, 50 μg/ml penicillin, and 100 μg/ml streptomycin) at 37° C. in an atmosphere of 5% CO₂.

The enrichment/culturing of transfected cells was effected under selective pressure with.100 μg/ml Zeocin.

| Cell line | Origin | Reference |
|---|---|---|
| 293T | Human embryonal kidney cells | (Graham, F. L. et al. 1977) |
| L540Cy | Hodgkin lymphoma | (Kapp, U. et al. 1992) |
| IMR5 | Neuroblastoma | (Bukovsky, J. et al. 1985) |

Table 1: Cell Lines Employed

Cloning Techniques

Recombinant Techniques

For the cloning, analysis and construction of the various DNA fragments and the plasmids employed, standard techniques were used (Sambrook, J. et al. 1989). The respective manufacturer's instructions for use of their products, especially for enzymes and kits, were suitably observed. Enzymes and kits supplied by Qiagen, Roche, NEB, AGS and Genecraft were used.

cDNA Preparation

Human RNA was obtained from whole blood using a QIAamp RNA Blood Mini Kit. The thus obtained RNA was transcribed into cDNA with the First-Strand cDNA Synthesis Kit supplied by Pharmacia Biotech. In addition to the primers provided in the kit, the specific primers for granzyme B were also used for first-strand synthesis.

PCR

The first-strand cDNA was immediately amplified in a PCR with the GB-specific primers. The design of the primers oriented itself by the sequence data available in the PubMed gene data base under the accession No. NM_{—}004131.

The PCR was performed under standard conditions in a primus thermocycler (MWG, Martinsried). Standard programming: 96° C., 5 min; 30×(96° C., 1 min; 60° C., 1 min; 72° C., 1 min); 72° C., 4 min.

Cloning of the Eukaryotic Expression Plasmids

The basic plasmid for the cloning and eukaryotic expression of the recombinant GB fusion proteins was pSecTag2 (Invitrogen, Netherlands). After various reclonings and modifications in the region of the MCS and marker epitopes, we were capable of cloning Ki-4(scFv) from the bacterial expression vector pBM1.1-Ki-4 (Barth, S. et al. 2000) and of cloning granzyme B into the newly designed pMS plasmids via Xho I/Cel II.

Further pMS plasmids derived therefrom contained the IVS and IRES sequences and the subsequent sequence for the reporter gene EGFP (green fluorescent protein) from the pIRES-EGFP plasmid (Clontech, USA). This enabled an uncomplicated determination of the transfection rate and simplified the selection of transfected cell populations.

Thus, all plasmids employed had the following features:

CMV promoter for constitutive expression of the GB constructs;

murine Ig-kappa leader for secreting the immunotoxins;

BGH-polyadenylation sequence;

Zeocin resistance gene for selection in eukaryotes;

ColE1-Ori for replication in prokaryotes;

ampicillin resistance gene for selection if prokaryotes.

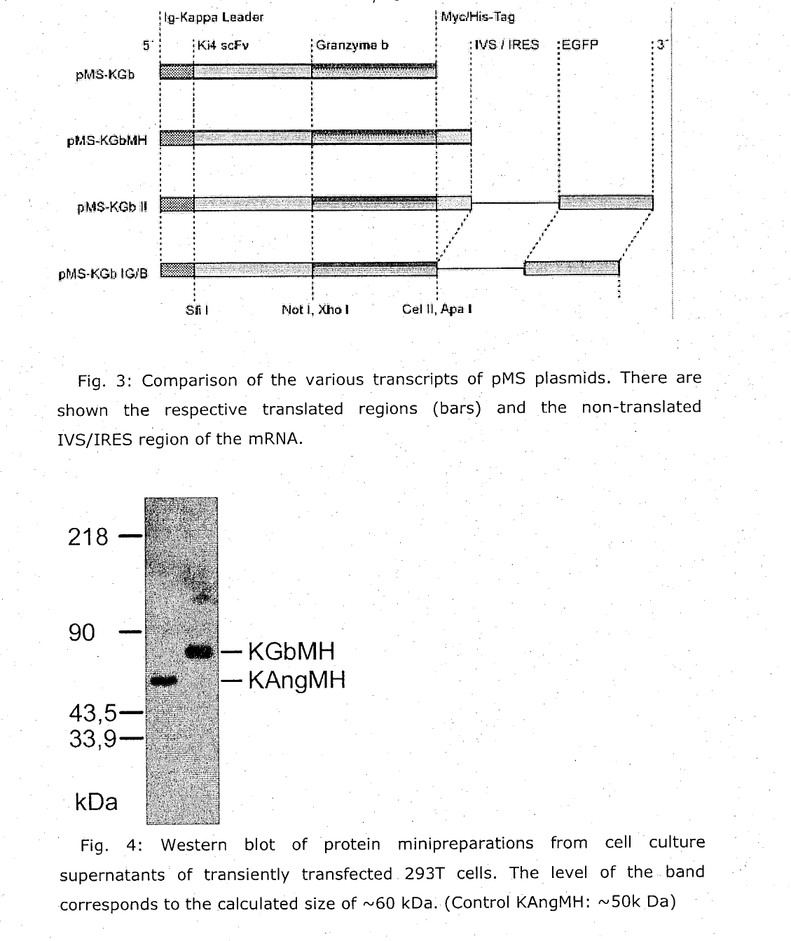

The differences between the plasmids employed are represented in FIG. 3. In contrast to pMS-KGbMH, the plasmid pMS-KGb codes for a Ki-4 (scFv)-granzyme B fusion protein without Myc and His tags and was intended to clarify whether sequences added to the C terminus influence the functionality of the immunotoxin. The addition of the IVS/IRES/EGFP sequence to these two constructs should clarify a possible effect of EGFP on the immunotoxin synthetic performance of the cells (e.g., pMS-KGb IG/B and pMS KGb II).

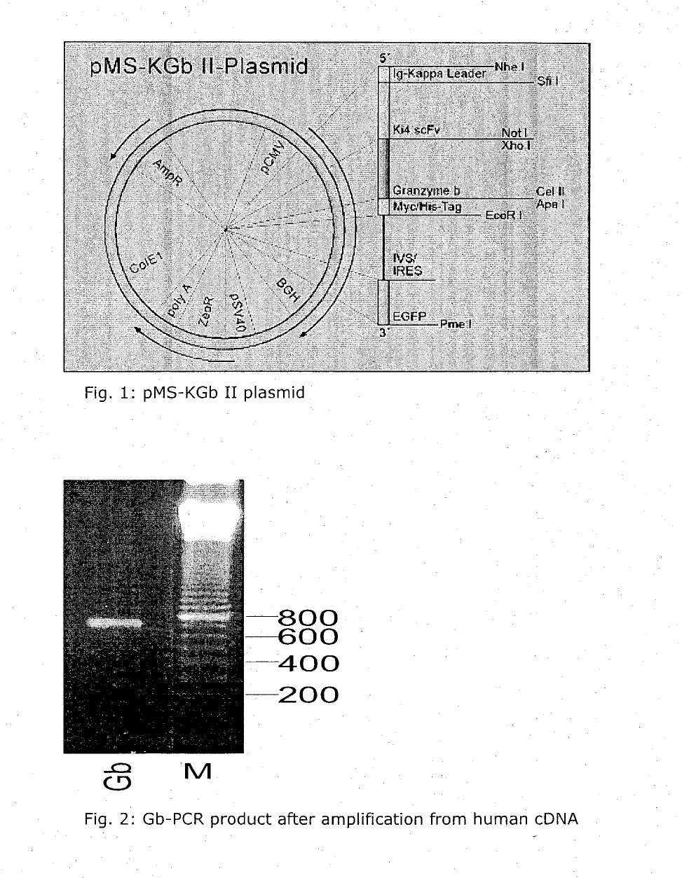

An example of the complete structure of the pMS plasmids is represented in FIG. 1.

Sequencing

The DNA sequencing was performed according to the dideoxy chain termination method (Sanger, F. et al. 1977). The employed ABI PRISM BigDye Terminator Cycle Sequencing Ready Reaction Kit contains all the necessary components for the reaction with the exception of templates and primers. The sequence reactions were performed on a Primus-96plus Thermocycler with a heating lid (MWG Biotech) without PCR oil.

Transfection of Eukaryotic Cells

The transfection of eukaryotic cells was performed with TransFast®, a synthetic cationic lipid (Promega). The plasmid employed, pMS-KGb II, comprised the EGFP reporter gene. The transfection rates for 293T cells were between 50 and 80%, which could be determined by counting the green fluorescing cells on the fluorescence cence microscope. The transfection was performed according to the manufacturer's protocol. After 3 days, the transfected cells were transferred into small cell culture jars and further cultured and selected under Zeocin® selective pressure (100 μg/ml).

Purification of the Recombinant Proteins

The protein purifications were performed exclusively with NiNTA (Qiagen). This method of immobilized metal affinity chromatography (IMAC) utilizes the affinity of histidine clusters (His tag) in proteins due to their charge for binding to Ni²⁺ ions immobilized through NTA (Hochuli, V. 1989; Porath, J. et al. 1975). Imidazole, a histidine analogue, competes with the His tag in the elution of the recombinant proteins.

Protein Minipreparation

The protein minipreparations were performed on the basis of the Qiagen protocols (The Expressionist July 1997) for the native purification of proteins with a His tag (Crowe, J. et al. 1994). The NiNTA was washed three times with 1× incubation buffer prior to use and stored therein at 4° C. (NiNTA 50%). The protocol was performed at room temperature, and all centrifugation steps were effected at 6000 rpm in a table-top centrifuge.

Centrifuge from 1.2 to 1.5 ml of cell culture supernatant for 2 min to sediment cells and cell components. To 900 μl of this cell culture supernatant, add 300 μl of 4× incubation buffer (200 mM NaH₂PO₄, pH 8.0; 1.2 M NaCl; 40 mM imidazole) and 30 μl of 50% NiNTA in a 1.5 ml Eppendorf vessel. Incubate for 1 h with shaking. Centrifuge for 1 min and discard the supernatant. Resuspend the NiNTA pellet two times in 175 μl of 1× incubation buffer and respectively discard the supernatants after centrifugation for 1 min. Add 30 μl of elution buffer (50 mM NaH₂PO₄, pH 8.0; 300 mM NaCl; 250 mM imidazole) to the NiNTA pellet and incubate at room temperature for 20 min with shaking. Centrifuge the pellet off for 1 min and transfer the supernatant with the purified protein into a new Eppendorf reaction vessel.

Purification of Proteins Through NiNTA Affinity Columns

Protein purification through NiNTA affinity columns was performed on a Bio-Rad Biologic Workstation with a fraction collector Model 2128 and an appropriate controller PC. The buffers employed are identical with those used in the protein minipreparation. After elution, the recombinant proteins were concentrated and rebuffered.

Protein Concentration and Rebuffering by Means of Ultrafiltration

In order to employ the purified proteins in various tests, the samples eluted from the NiNTA column had to be concentrated, their concentrations determined, and rebuffered. The rebuffering in PBS also removed the imidazole of the elution buffer, which is harmful to cells, from the preparations.

For concentration and rebuffering, an Amicon 2000 ultrafiltration chamber and Diaflo ultrafiltration membranes with a pore exclusion size for proteins of <10 kDa were employed. Under high pressure from a nitrogen gas cylinder, the GB fusion proteins were concentrated and subsequently rebuffered.

After sterilization by filtration, the concentrated protein solution was stored at 4° C. in a 1.5 ml reaction vessel.

Determination of Protein Quantities

To determine the total protein concentration of the concentrated protein samples, a modified Lowry assay (Lowry, O. H. et al. 1951) was used (Bio-Rad DC Protein Test).

In addition to the total protein determination, an SDS-PAGE gel electrophoresis is performed with the samples, followed by Coomassie staining of the gel. This enabled an estimation of the proportion of purified recombinant protein in the total protein in the sample.

SDS Polyacrylamide Gel Electrophoresis (SDS-PAGE)

For the gel electrophoresis of proteins, there were exclusively used prefabricated linear 4-15% Tris-HCI gradient gels in the corresponding Ready Gel Cell (Bio-Rad). After boiling the samples in non-reducing 4× Roti-Load sample buffer (Roth) for 10 min, the samples were applied to the gels. Gel electrophoresis was performed in a Tris/glycine/SDS running buffer (Bio-Rad) for 0.6 h with 200 V at room temperature.

Western Blot

A Western blot was performed by the tank method in a Mini Trans Blot Cell (Bio-Rad) on PVDF-Hybond membranes (Amersham/Pharmacia). Transfer conditions: 1.2 h at 500 mA in blotting buffer (25 mM Tris-base; 192 mM glycine, pH 8.3; 20% methanol).

Immunostaining of Western Blots

The immunostaining of the blotted proteins was performed according to standard methods. The detection of the GB fusion proteins was effected with the Qiagen anti-penta-His antibody (1/5000 vol. in TTBS (1.4 g/l Tris-base; 6.05 g/l Tris/HCl; 8.78 g/l NaCl, pH 7.5; 0.05% Tween 20; 0.1% BSA)). The detection was performed formed through an HRP-conjugated donkey anti-mouse IgG (Dianova) (1/10,000 vol. in TTBS). For the final chemiluminescence reaction, the ECL system (Amersham Pharmacia) was used, and appropriate X-ray films (Roche) were exposed.

Coomassie Staining of SDS-PAGE Gels

SDS-PAGE gels were placed into the staining solution (0.25% Coomassie Brilliant Blue R250; 45% methanol; 45% ddH₂O; 10% acetic acid) and incubated on a rotary shaker for 1 h. Then, the SDS-PAGE gels were repeatedly washed in a decoloring solution (45% methanol; 45% ddH₂O; 10% acetic acid) and finally purified with H₂O.

In-Vitro Characterization of the Recombinant Proteins

FACS Analyses

The binding capacity of the KGb constructs secreted by the transfected cells was determined by cell flow cytometry (Barth, S. et al. 1998). Cell suspensions with 2×10⁵ cells were shortly washed in PBS/BSA/N₃ (PBS with 0.2% BSA and 0.05% sodium azide) and subsequently incubated with cell culture supernatants of purified GB apoptotic agents in PBS/BSA/N₃ for 30 min at 4° C. After 3 washings, the cells were incubated for 30 min at 4° C. with 1/1000 vol. of anti-penta-His in PBS/BSA/azide. Then, the cells were again washed three times and incubated for 15 min with 1/50 vol. goat anti-mouse Ab in PBS/BSA/azide. After 3 more washings in PBS/BSA/azide, the cell suspension was admixed with 2 μl of 6.25 mg/ml propidium iodide and immediately analyzed in a FACS-Calibur (Becton Dickinson, Heidelberg, Germany).

XTT Viability Tests

The determination of the cytotoxic potential of the GB fusion proteins was determined through the substrate conversion of yellow tetrazolium salt to water-soluble formazane dye by cells (Barth, S. et al. 2000). The relative viability of the cells was determined using positive controls of cells treated with Zeocin.

In 96-well flat-bottomed plates, serial dilutions of the toxin or of cell culture supernatants were respectively employed in duplicate to quadruplicate. Thus, 120 μl of supernatant was added to each well of the first row, and 100 μl of complex medium was added to the remaining wells of the serial dilutions. Pipette 20 μl each from the first row into the next dilution stage (1:5 dilution). Positive control: 120 μl of complex medium with Zeocin (100 to 200 μg/ml); negative control: only complex medium. Then, from 1 to 0.8×10⁴ cells in 100 μl of complex medium were pipetted to the serial dilutions, followed by incubation for 24-48 h at 37° C. in an incubator at 5% CO₂. After the addition of 50 μl of XTT/PMS (final concentration of 0.3 mg and 0.383 ng) per well and another incubation for 4-48 h of the cells, a photometric measurement of the XTT substrate conversion was performed as a subtraction of OD_{450 nm}−OD_{650 nm} in an ELISA reader.

Results

Granzyme B PCR

In addition to the GB-specific sequences (capital letters), restriction sites for further cloning were added to the amplification product through the primers (xx-Gb-back: XbaI, XhoI; Gb-for: Cel II, BamHI).

xx-Gb-back:
gcactcgagtctagaATCATCGGGGGACATGAGGCCAAG
Gb-for:
ttcgtgctcagctagtttggatccGTAGCGTTTCATGGTTTTCTTTATC

The product of the PCR showed the expected length of 720 bp (FIG. 2).

Plasmid Constructions

All clonings of the GB were performed through Xho I and Cel II into the various available pMS plasmids. Verification of the clonings was effected by specific restriction analyses, sequencing of KGbII and the immunohistochemical detection of KGbMH in the supernatant of transfected 293T cells (e.g., pMS-KGbMH and pMS-KGb II) (FIG. 3).

Sequencing

A first sequencing was performed on the GB-PCR product with the GB-specific primers and confirmed the GB sequence. The complete GB sequence was established on the basis of the pMS-KGb II plasmid. For overlapping sequencing in both directions, there were respectively employed the GB-specific primers (e.g., xx-Gb-back; Gb-for) and one plasmid-located primer each about 100 bp 5′ or 3′ from the restriction sites relevant to cloning. This sequencing showed 100% homology with the GB sequence published in the gene data base of PubMed under the accession No. NM_{—}004131.

Expression of the Recombinant Proteins

The expression of the apoptotic agents was effected exclusively in eukaryotic cells (e.g., 293T). FIG. 4 shows the result of a Western blot after a protein minipreparation.

FACS Analyses

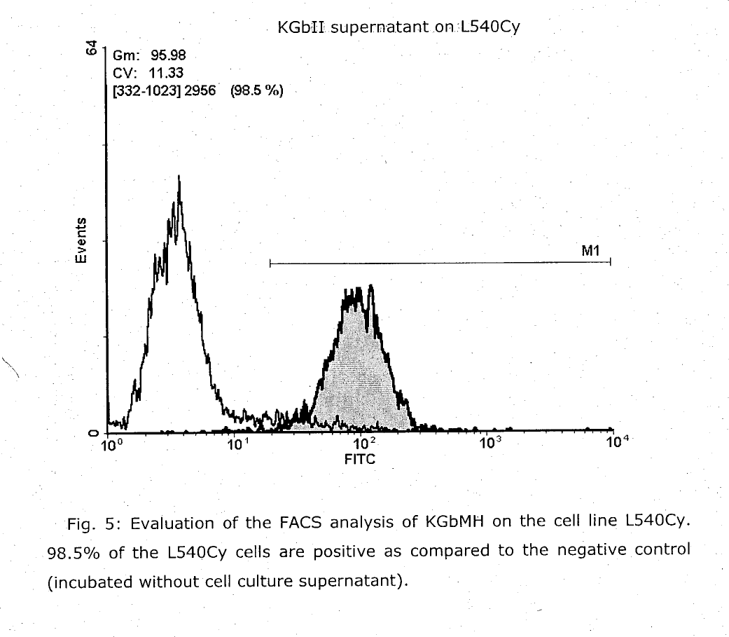

To evaluate the binding capability of the KGb apoptotic agents expressed in eukaryotes, FACS analyses were performed for CD30(+) (e.g., L540Cy) and CD30(−) cell lines (e.g., IMR5). On the negative cell line and the corresponding controls, no staining of the cells could be documented in the FACS. In contrast, the staining of the CD30(+) cells with the KGb apoptotic agents was identical with that of the positive control with Ki4moAb (FIG. 5). This demonstrated the Ki-4(scFv)-mediated binding of the KGbMH apoptotic agents and the lack of non-specific binding to the examined cells.

XTT Viability Tests

Competition of KGbMH with Ki4-moAb

In addition to the FACS analyses, a competition of the KGbMH with the monoclonal Ki-4 antibody was performed on L540Cy. In addition to a simple XTT viability test with cell culture supernatants (KGb II) of stably transfected 293T cells, a serial dilution of Ki4-moAb (initial concentration: 40 μg/ml) was employed in 100 μl of a KGbMH-containing cell culture supernatant (KG+Ki4). The mirror-symmetrical course of the viability curves in FIG. 6 shows that KGbMH can be successfully blocked by Ki-4-moAb.

Supernatants of Transiently Transfected 293T Cells

The XTT viability test with the cell culture supernatants of transiently transfected 293T cells served for the clarification of central questions:

Are human/eukaryotic cells capable of producing and secreting an apoptotic agent which is potentially toxic towards them?

Is the GB apoptotic agent synthetic performance of the transfected cells sufficient to display a cytotoxic effect on the target cells, and is thus a potential use in gene therapy possible?

How does the C-terminal addition of marker epitopes (e.g., Myc and His tag) influence the functionality of the GB apoptotic agents?

Is GB apoptotic agent synthesis adversely affected by the simultaneous synthesis of the EGFP?

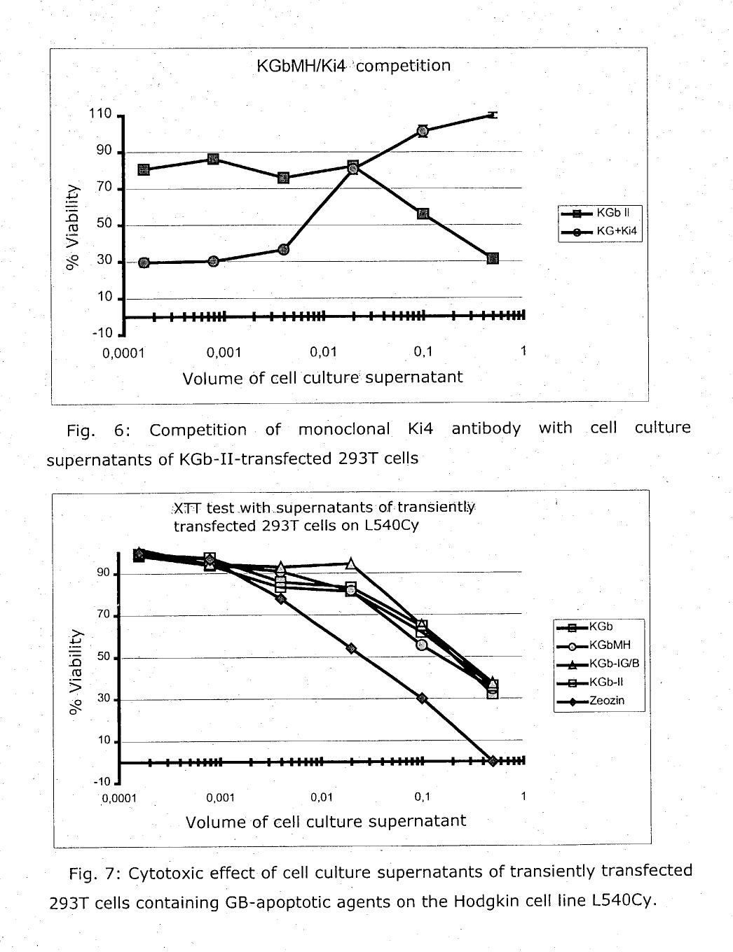

In a 12-well plate with TransFast, 1×10⁵ 293T cells each were transfected with 1 μg each of plasmid DNA and 3 μl of TransFast. Transfections were performed in duplicate. After 72 h from the transfection, the cell culture supernatants were employed at 120 μl each in the first dilution stage of an XTT viability test (4 rows/construct). The measurement was performed 48 h after the addition of XTT/phenazine. The evaluation of the viability test is represented in FIG. 7.

The represented results show that neither the C-terminal modifications performed nor the simultaneous expression of the EGFP reporter gene has a remarkable influence on the functionality or quantity of the secreted GB apoptotic agents.

In addition, the results suggest that plasmids coding for and secreting GB apoptotic agents may potentially find use also within the scope of a gene therapy.

In addition to the XTT viability tests on L540Cy, controls with the CD30-negative cell line IMR5 were also performed. In this case, a cytotoxic effect of the KGbMH apoptotic agents on the cells could not be observed.

Determination of the IC₅₀ of KGbMH

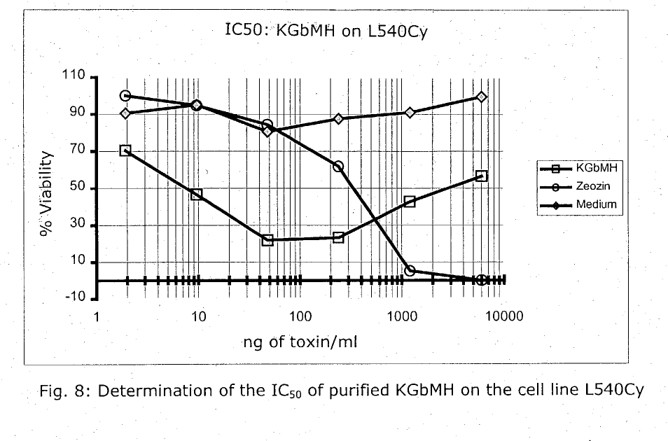

After purification of KGbMH from cell culture supernatants of cells transfected with pMS-KGb II and after a FACS analysis, protein quantity determination and purification estimation of the preparation, an XTT viability test with L540Cy was performed using a Coomassie gel. The IC₅₀ determined from the graphical evaluation of the data in FIG. 8 is 7.5 ng/ml for the KGbMH. This order of magnitude approximately corresponds to that of classical immunotoxins, such as Ki-4 (scvFv)-ETA′ (3-6 ng/ml) (Barth, S. et al. 2000).

References

Andrade, F., Roy, S., Nicholson, D., Thornberry, N., Rosen, A. and Casciola-Rosen, L. Granzyme B directly and efficiently cleaves several downstream caspase substrates: implications for CTL-induced apoptosis. Immunity 8(4): 451-60 (1998).

Baluna, R. and Vitetta, E. S. Vascular leak syndrome: a side effect of immunotherapy therapy. Immunopharmacology 37(2-3): 117-132 (1997).

Barth, S., Huhn, M., Matthey, B., Klimka, A., Galinski, E. A. and Engert, A. Compatible-solute-supported periplasmic expression of functional recombinant proteins under stress conditions. Appl Environ Microbiol 66(4): 1572-1579 (2000).

Barth, S., Huhn, M., Matthey, B., Klimka, A., Tawadros, S., Schnell, R., Diehl, V. and Engert, A. Ki-4(scFv)-ETA′, a new recombinant anti-CD30 immunotoxin with highly specific cytotoxic activity against disseminated Hodgkin tumors in SCID mice. Blood 95: 3909-3914 (2000).

Barth, S., Huhn, M., Matthey, B., Tawadros, S., Schnell, R., Schinkothe, T., Diehl, V. and Engert, A. Ki-4(scFv)-ETA′, a new recombinant anti-CD30 immunotoxin with highly specific cytotoxic activity against disseminated Hodgkin tumors in SCID mice. Blood 95(12): 3909-14 (2000).

Barth, S., Huhn, M., Wels, W., Diehl, V. and Engert, A. Construction and in vitro evaluation of RFT5(scFv)-ETA′, a new recombinant single-chain immunotoxin with specific cytotoxicity toward CD25+ Hodgkin-derived cell lines. Int J Mol Med 1(1): 249-256 (1998).

Battelli, M. G., Buonamici, L., Polito, L., Bolognesi, A. and Stirpe, F. Hepatoxicity of ricin, saporin or a saporin immunotoxin: xanthine oxidase activity in rat liver and blood serum. Virchows Arch 427(5): 529-35 (1996).

Beaumelle, B., Bensammar, L. and Bienvenue, A. Selective translocation of the A chain of diphtheria toxin across the membrane of purified endosomes. J Biol Chem 267(16): 11525-11531 (1992).

Beresford, P. J., Xia, Z., Greenberg, A. H. and Lieberman, J. Granzyme A loading induces rapid cytolysis and a novel form of DNA damage independently of caspase activation [published erratum appears in Immunity June 1999; 10(6):following 768]. Immunity 10(5): 585-94 (1999).

Bolognesi, A., Tazzari, P. L., Olivieri, F., Polito, L., Falini, B. and Stirpe, F. Induction of apoptosis by ribosome-inactivating proteins and related immunotoxins. Int J Cancer 68(3): 349-55 (1996).

Brenner, T., Steinberger, I., Soffer, D., Beraud, E., Ben-Nun, A. and Lorber-boum-Galski, H. A novel antigen-toxin chimeric protein: myelin basic protein-pseudomonas exotoxin (MBP-PE 40) for treatment of experimental autoimmune encephalomyelitis. Immunol Lett 68(2-3): 403-10 (1999).

Bukovsky, J., Evans, A., Tartaglione, M. and Kennett, R. H. Selection of variant neuroblastoma cell line which has lost cell surface expression of antigen detected by monoclonal antibody PI153/3. Somat Cell Mol Genet 11(5): 517-22 (1985).

Chaudhary, V., Batra, J., Gallo, M., Willingham, M., Fitz, G. D. and Pastan, I. A rapid method of cloning functional variable-region antibody genes in Escherichia coli as single-chain immunotoxins [published erratum appears in Proc Natl Acad Sci USA April 1990; 87(8):3253]. Proc Natl Acad Sci USA 87(3): p1066-70 (1990).

Chaudhary, V., Gallo, M., Fitz, G. D. and Pastan, I. A recombinant single-chain immunotoxin composed of anti-Tac variable regions and a truncated diphtheria toxin. Proc Natl Acad Sci USA 87(23): p9491-4 (1990).

Chaudhary, V., Queen, C., Junghans, R., Waldmann, T., Fitz, G. D. and Pastan, I. A recombinant immunotoxin consisting of two antibody variable domains fused to Pseudomonas exotoxin. Nature 339(6223): p394-7 (1989).

Chen, S., Yang, A., Chen, J., Kute, T., King, C., Collier, J., Cong, Y., Yao, C. and Huang, X. Potent antitumour activity of a new class of tumour-specific killer cells. Nature 385(6611): p78-80 (1997).

Crowe, J., Dobeli, H., Gentz, R., Hochuli, E., Stuber, D. and Henco, K. 6×His-Ni-NTA chromatography as a superior technique in recombinant protein expression/purification. Methods Mol Biol 31: 371-87 (1994).

Endo, Y., Mitsui, K., Motizuki, M. and Tsurugi, K. The mechanism of action of ricin and related toxic lectins on eukaryotic ribosomes. The site and the characteristics teristics of the modification in 28 S ribosomal RNA caused by the toxins. J Biol Chem 262(12): 5908-12 (1987).

Engert, A., Diehl, V., Schnell, R., Radszuhn, A., Hatwig, M. T., Drillich, S., Schon, G., Bohlen, H., Tesch, H., Hansmann, M. L., Barth, S., Schindler, J., Ghetie, V., Uhr, J. and Vitetta, E. A phase-I study of an anti-CD25 ricin A-chain immunotoxin (RFT5-SMPT-dgA) in patients with refractory Hodgkin's lymphoma. Blood 89(2): 403-410 (1997).

Fernandes-Alnemri, T., Armstrong, R. C., Krebs, J., Srinivasula, S. M., Wang, L., Bullrich, F., Fritz, L. C., Trapani, J. A., Tomaselli, K. J., Litwack, G. and Alnemri, E. S. In vitro activation of CPP32 and Mch3 by Mch4, a novel human apoptotic cysteine protease containing two FADD-like domains. Proc Natl Acad Sci USA 93(15): 7464-9 (1996).

Fett, J. W., Strydom, D. J., Lobb, R. R., Alderman, E. M., Bethune, J. L., Riordan, J. F. and Vallee, B. L. Isolation and characterization of angiogenin, an angiogenic protein from human carcinoma cells. Biochemistry 24(20): 5480-6 (1985).

FitzGerald, D. J., Willingham, M. C. and Pastan, I. Pseudomonas exotoxin—immunotoxins. Cancer Treat Res 37: 161-173 (1988).

Graham, F. L., Smiley, J., Russell, W. C. and Nairn, R. Characteristics of a human cell line transformed by DNA from human adenovirus type 5. J Gen Virol 36(1): 59-74 (1977).

Greenberg, A. H. Granzyme B-induced apoptosis. Adv Exp Med Biol 406: 219-28 (1996).

Grossbard, M. L., Fidias, P., Kinsella, J., O'Toole, J., Lambert, J. M., Blattler, W. A., Esseltine, D., Braman, G., Nadler, L. M. and Anderson, K. C. Anti-B4-blocked ricin: a phase II trial of 7 day continuous infusion in patients with multiple myeloma. Br J Haematol 102(2): 509-515 (1998).

Grossbard, M. L., Lambert, J. M., Goldmacher, V. S., Spector, N. L., Kinsella, J., Eliseo, L., Coral, F., Taylor, 3. A., Blattler, W. A. and Epstein, C. L. Anti-B4-blocked ricin: a phase I trial of 7-day continuous infusion in patients with B-cell neoplasms. J Clin Oncol 11(4): 726-737 (1993).

Harkonen, S., Stoudemire, J., Mischak, R., Spitler, L. E., Lopez, H. and Scannon, P. Toxicity and immunogenicity of monoclonal antimelanoma antibody-ricin A chain immunotoxin in rats. Cancer Res 47(5): 1377-82 (1987).

Hochuli, V. Cholesterol testing for all [letter]. Bmj 298(6677): 891 (1989).

Iglewski, B. H. and Kabat, D. NAD-dependent inhibition of protein synthesis by Pseudomonas aeruginosa toxin. Proc Natl Acad Sci USA 72(6): 2284-8 (1975).

Jans, D. A., Jans, P., Briggs, L. J., Sutton, V. and Trapani, J. A. Nuclear transport of granzyme B (fragmentin-2). Dependence of perforin in vivo and cytosolic factors in vitro. J Biol Chem 271(48): 30781-9 (1996).

Jurcic, J. G. and Scheinberg, D. A. Radioimmunotherapy of hematological cancer: problems and progress. Clin Cancer Res 1(12): 1439-1446 (1995).

Kam, C. M., Hudig, D. and Powers, J. C. Granzymes (lymphocyte serine proteases): characterization with natural and synthetic substrates and inhibitors. Biochim Biophys Acta 1477(1-2): 307-23 (2000).

Kaminski, M. S., Zasadny, K. R., Francis, I. R., Fenner, M. C., Ross, C. W., Milik, A. W., Estes, J., Tuck, M., Regan, D., Fisher, S., Glenn, S. D. and Wahi, R. L. Iodine-131-anti-B1 radioimmunotherapy for B-cell lymphoma. J Clin Oncol 14(7): 1974-1981 (1996).

Kapp, U., Wolf, J., von Kalle, C., Tawadros, S., Rottgen, A., Engert, A., Fonatsch, C., Stein, H. and Diehl, V. Preliminary report: growth of Hodgkin's lymphoma derived cells in immune compromised mice. Ann Oncol 3 Suppl 4: 21-23 (1992).

Keppler-Hafkemeyer, A., Brinkmann, U. and Pastan, I. Role of caspases in immunotoxin-induced apoptosis of cancer cells. Biochemistry 37(48): 16934-16942 (1998).

Keppler-Hafkemeyer, A., Kreitman, R. J. and Pastan, I. Apoptosis induced by immunotoxins used in the treatment of hematologic malignancies. Int J Cancer 87(1): 86-94 (2000).

Khazaeli, M. B., Conry, R. M. and Lo Buglio, A. F. Human immune response to monoclonal antibodies. J Immunother 15(1): 42-52 (1994).

Klimka, A., Barth, S., Drillich, S., Wels, W., van Snick, J., Renauld, J. C., Tesch, H., Bohlen, H., Diehl, V. and Engert, A. A deletion mutant of Pseudomonas exotoxin-A fused to recombinant human interleukin-9 (rhIL-9-ETA′) shows specific cytotoxicity against IL-9-receptor-expressing cell lines. Cytokines Mol Ther 2(3): 139-146 (1996).

Kreitman, R. J., Wilson, W. H., White, J. D., Stetler-Stevenson, M., Jaffe, E. S., Giardina, S., Waldmann, T. A. and Pastan, I. Phase I trial of recombinant immunotoxin anti-Tac(Fv)-PE38 (LMB-2) in patients with hematologic malignancies. J Clin Oncol 18(8): 1622-1636 (2000).

Kuzel, T. M., Rosen, S. T., Gordon, L. I., Winter, J., Samuelson, E., Kaul, K., Roenigk, H. H., Nylen, P. and Woodworth, T. Phase I trial of the diphtheria toxin/interleukin-2 fusion protein DAB486IL-2: efficacy in mycosis fungoides and other non-Hodgkin's lymphomas. Leuk Lymphoma 11(5-6): 369-377 (1993).

Leland, P. A., Schultz, L. W., Kim, B. M. and Raines, R. T. Ribonuclease A variants with potent cytotoxic activity. Proc Natl Acad Sci USA 95(18): 10407-12 (1998).

LeMaistre, C., Saleh, M., Kuzel, T., Foss, F., Platanias, L., Schwartz, G., Ratain, M., Rook, A., Freytes, C., Craig, F., Reuben, J. and Nichols, J. Phase I trial of a ligand fusion-protein (DAB389IL-2) in lymphomas expressing the receptor for interleukin-2. Blood 91(2): p399-405. (1998).

LeMaistre, C. F., Meneghetti, C., Rosenblum, M., Reuben, J., Parker, K., Shaw, J., Deisseroth, A., Woodworth, T. and Parkinson, D. R. Phase I trial of an interleukin-2 (IL-2) fusion toxin (DAB486IL-2) in hematologic malignancies expressing the IL-2 receptor. Blood 79(10): 2547-2554 (1992).

LoBuglio, A. F. and Saleh, M. N. Monoclonal antibody therapy of cancer. Crit Rev Oncol Hematol 13(3): 271-82 (1992).

Lowry, O. H., Rosebrough, N. J., Farr, A. L. and Randall, R. J. Protein Measurement with the Folin Phenol Reagent. Journal of Biological Chemistry 193: 265-275 (1951).

Matthey, B., Engert, A. and Barth, S. Ki-4(scFv )-Ricin, a new recombinant immunotoxin targeting CD30 isolated from E.coli periplasmic space exhibits specific cytotoxic activity against Hodgkin cells in vitro. submitted (2000).

Newton, D., Nicholls, P., Rybak, S. and Youle, R. Expression and characterization of recombinant human eosinophil-derived neurotoxin and eosinophil-derived neurotoxin-anti-transferrin receptor sFv. J Biol Chem 269(43): p26739-45 (1994).

Newton, D., Xue, Y., Olson, K., Fett, J. and Rybak, S. Angiogenin single-chain immunofusions: influence of peptide linkers and spacers between fusion protein domains. Biochemistry 35(2): p545-53 (1996).

Newton, D. L., Ilercil, O., Laske, D. W., Oldfield, E., Rybak, S. M. and Youle, R. J. Cytotoxic ribonuclease chimeras. Targeted tumoricidal activity in vitro and in vivo. J Biol Chem 267(27): 19572-8 (1992).

Nicholson, D. W. and Thornberry, N. A. Caspases: killer proteases. Trends Biochem Sci 22(8): 299-306 (1997).

O'Hare, M., Brown, A. N., Hussain, K., Gebhardt, A., Watson, G., Roberts, L. M., Vitetta, E. S., Thorpe, P. E. and Lord, J. M. Cytotoxicity of a recombinant ricin-A-chain fusion protein containing a proteolytically-cleavable spacer sequence. FEBS Lett 273(1-2): 200-4 (1990).

Pai, L. H. and Pastan, I. Clinical trials with Pseudomonas exotoxin immunotoxins. Curr Top Microbiol Immunol 234: 83-96 (1998).

Pinkoski, M. J., Heibein, J. A., Barry, M. and Bleackley, R. C. Nuclear translocation tion of granzyme B in target cell apoptosis. Cell Death Differ 7(1): 17-24 (2000).

Pirker, R. Immunotoxins against solid tumors. J Cancer Res Clin Oncol 114(4): 385-93 (1988).

Porath, J., Carlsson, J., Olsson, I. and Belfrage, G. Metal chelate affinity chromatography, tography, a new approach to protein fractionation. Nature 258(5536): 598-9 (1975).

Press, O. W., Eary, J., Badger, C. C., Appelbaum, F. R., Wiseman, G., Matthews, D., Martin, P. J. and Bernstein, I. D. High-dose radioimmunotherapy of lymphomas. Cancer Treat Res 68: 13-22 (1993).

Rybak, S., Hoogenboom, H., Meade, H., Raus, J., Schwartz, D. and Youle, R. Humanization of immunotoxins. Proc Natl Acad Sci USA 89(8): p3165-9 (1992).

Rybak, S., Saxena, S., Ackerman, E. and Youle, R. Cytotoxic potential of ribonuclease and ribonuclease hybrid proteins. J Biol Chem 266(31): p21202-7 (1991).

Saleh, M. N., Khazaeli, M. B., Wheeler, R. H., Dropcho, E., Liu, T., Urist, M., Miller, D. M., Lawson, S., Dixon, P., Russell, C. H. and et al. Phase I trial of the murine monoclonal anti-GD2 antibody 14G2a in metastatic melanoma. Cancer Res 52(16): 4342-7 (1992).

Sambrook, J., Fritsch, E. and Maniatis, T. (1989). Molecular cloning. A laboratory manual. New York, Cold Spring Harbor Laboratory Press.

Sanger, F., Nicklen, S. and Coulson, A. R. DNA sequencing with chain-terminating inhibitors. Proc Natl Acad Sci USA 74(12): 5463-5467 (1977).

Sarin, A., Williams, M. S., Alexander-Miller, M. A., Berzofsky, J. A., Zacharchuk, C. M. and Henkart, P. A. Target cell lysis by CTL granule exocytosis is independent of ICE/Ced-3 family proteases. Immunity 6(2): 209-15 (1997).

Saxena, S. K., Rybak, S. M., Davey, R. T. J., Youle, R. J. and Ackerman, E. J. Angiogenin is a cytotoxic, tRNA-specific ribonuclease in the RNase A superfamily. J Biol Chem 267(30): 21982-21986 (1992).

Schnell, R., Vitetta, E., Schindler, J., Barth, S., Winkler, U., Borchmann, P., Hansmann, M. L., Diehl, V., Ghetie, V. and Engert, A. Clinical trials with an anti-CD25 ricin A-chain experimental and immunotoxin (RFT5-SMPT-dgA) in Hodgkin's lymphoma. Leuk Lymphoma 30(5-6): 525-537 (1998).

Schnell, R., Vitetta, E., Schindler, J., Borchmann, P., Barth, S., Ghetie, V., Hell, K., Drillich, S., Diehl, V. and Engert, A. Treatment of refractory Hodgkin's lymphoma patients with an anti-CD25 ricin A-chain immunotoxin. Leukemia 14(1): 129-135 (2000).

Shapiro, R., Riordan, J. F. and Vallee, B. L. The characteristic ribonuclease activity of angiogenin. Biochem. Insert 25: 3527-3532 (1986).

Shresta, S., Pham, C. T., Thomas, D. A., Graubert, T. A. and Ley, T. J. How do cytotoxic lymphocytes kill their targets? Curr Opin Immunol 10(5): 581-7 (1998).

Srinivasula, S. M., Fernandes-Alnemri, T., Zangrilli, 3., Robertson, N., Armstrong, R. C., Wang, L., Trapani, J. A., Tomaselli, K. J., Litwack, G. and Alnemri, E. S. The Ced-3/interleukin lbeta converting enzyme-like homolog Mch6 and the lamin-cleaving enzyme Mch2alpha are substrates for the apoptotic mediator CPP32. J Biol Chem 271(43): 27099-106 (1996).

Sutton, V. R., Davis, J. E., Cancilla, M., Johnstone, R. W., Ruefli, A. A., Sedelies, K., Browne, K. A. and Trapani, J. A. Initiation of apoptosis by granzyme B requires direct cleavage of bid, but not direct granzyme B-mediated caspase activation [In Process Citation]. J Exp Med 192(10): 1403-14 (2000).

Thorpe, P. E., Wallace, P. M., Knowles, P. P., Relf, M. G., Brown, A. N., Watson, G. J., Blakey, D. C. and Newell, D. R. Improved antitumor effects of immunotoxins prepared with deglycosylated ricin A-chain and hindered disulfide linkages. Cancer Res 48(22): 6396-6403 (1988).

Trapani, J. A., Jans, D. A., Jans, P. J., Smyth, M. J., Browne, K. A. and Sutton, V. R. Efficient nuclear targeting of granzyme B and the nuclear consequences of apoptosis induced by granzyme B and perforin are caspase-dependent, but cell death is caspase-independent. J Biol Chem 273(43): 27934-8 (1998).

Trapani, J. A., Jans, P., Smyth, M. J., Froelich, C. J., Williams, E. A., Sutton, V. R. and Jans, D. A. Perforin-dependent nuclear entry of granzyme B precedes apoptosis, and is not a consequence of nuclear membrane dysfunction. Cell Death Differ 5(6): 488-96 (1998).

Vitetta, E. S. Immunotoxins and vascular leak syndrome [In Process Citation]. Cancer J Sci Am 6 Suppi 3: S218-224 (2000).

Yokota, T., Milenic, D. E., Whitlow, M. and Schlom, J. Rapid tumor penetration of a single-chain Fv and comparison with other immunoglobulin forms. Cancer Res 52(12): 3402-3408 (1992).

Yoon, J. M., Han, S. H., Kown, O. B., Kim, S. H., Park, M. H. and Kim, B. K. Cloning and cytotoxicity of fusion proteins of EGF and angiogenin. Life Sci 64(16): 1435-1445 (1999).

Youle, R. 3. and Neville, D. M. J. Anti-Thy 1.2 monoclonal antibody linked to ricin is a potent cell-type-specific toxin. Proc Natl Acad Sci USA 77(9): 5483-5486 (1980).

Zewe, M., Rybak, S., Dubel, S., Coy, J., Welschof, M., Newton, D. and Little, M. Cloning and cytotoxicity of a human pancreatic RNase immunofusion. Immunotechnology 3(2): p127-36 (1997).

1. A complex formed from at least one component A and at least one component B, characterized in that component A has a binding activity for cellular surface structures, and component B is a granzyme.

2. The complex according to claim 1, containing one or more supplementary components S in addition to said components A and B.

3. The complex according to claims 1 and/or 2, characterized in that component A is selected from the group of actively binding structures consisting of antibodies or their derivatives or fragments, synthetic peptides or chemical molecules, ligands, lectins, receptor binding molecules, cytokines, lymphokines, chemokines, adhesion molecules and their derivatives, mutants or combinations thereof, which bind to cluster of differentiation antigens, cytokine, hormone, growth factor receptors, ion pumps, channel-forming proteins.

4. The complex according to claims 1 and 2, characterized in that component A is selected from the group of passively bound structures consisting of allergens, peptidic allergens, recombinant allergens, allergen-idiotypical antibodies, autoimmune-provoking structures, tissue-rejection-inducing structures and their derivatives, mutants or combinations thereof.

5. The complex according to claims 1 to 4, characterized in that component B has proteolytic properties or is at least one protease, their derivatives, mutant or combination.

6. The complex according to claims 1 to 5, characterized in that component B directly activates components of cell-inherent apoptosis and thus induces apoptosis in the cells defined through the binding of component A.

7. The complex according to claims 1 to 6, characterized in that component B is granzyme B or a derivative thereof.

8. The complex according to claims 1 to 7, characterized in that a component S regulates the synthetic performance.

9. The complex according to claims 1 to 8, characterized in that a component S regulates the synthetic performance.

10. The complex according to claim 8, characterized in that said component S enables the purification of the complex or its components.

11. The complex according to claims 8 to 10, characterized in that said component S supports the translocation into target cells.

12. The complex according to claims 8 to 11, characterized in that said component S enables intracellular activation of component B.

13. Nucleic acid molecules or vectors coding for a complex designed according to claims 1 to 11 for or individual components for the preparation of such complex.

14. Cellular compartments or organisms, characterized by synthesizing complete complexes according to any of claims 1 to 11 or individual components thereof after having been transformed or transfected with nucleic acid molecules or vectors according to claim 12.

15. The cellular compartments according to claim 13, characterized by being of either prokaryotic origin, especially from E. coli, B. subtilis, S. carnosus, S. coelicolor, Marinococcus sp., or eukaryotic origin, especially from Saccharomyces sp., Aspergillus sp., Spodoptera sp., P. pastoris, primary or cultivated mammal cells, eukaryotic cell lines (e.g., CHO, Cos or 293) or plant systems (e.g. N. tabacum).

16. A medicament containing a complex according to any of claims 1 to 11, nucleic acid molecules according to claim 12, or cellular compartments according to either of claims 13 and/or 14.

17. Use of the complex according to any of claims 1 to 11, of the nucleic acid molecules coding therefor according to claim 12, and/or of cellular compartments according to claims 13 and/or 14, for treating malignant diseases, eases, allergies, autoimmune reactions, chronic inflammation reactions or tissue rejection reactions.


SEQUENCE LISTING (2 sequences)

SEQ ID NO: 1 - DNA, 39 bp, Artificial Sequence
  Description of Artificial Sequence Primer xx-Gb-back for Cloning
gcactcgagtctagaatcatcgggggacatgaggccaag

SEQ ID NO: 2 - DNA, 49 bp, Artificial Sequence
  Description of Artificial Sequence Primer Gb-for for Cloning
ttcgtgctcagctagtttggatccgtagcgtttcatggttttctttatc